(19) United States
(12) Patent Application Publication
(10) Pub. No.: US 2022/0184052 A1
(43) Pub. Date: Jun. 16, 2022

(54) COMPOSITION COMPRISING FEXOFENADINE

(71) Applicant: Cellix Bio Private Limited, Hyderabad (IN)

(72) Inventor: Mahesh Kandula, Andhra Pradesh (IN)

(73) Assignee: Cellix Bio Private Limited, Hyderabad (IN)

(21) Appl. No.: 17/650,663

(22) Filed: Feb. 10, 2022

Related U.S. Application Data
(63) Continuation of application No. PCT/IB2021/061561, filed on Dec. 10, 2021.

(30) Foreign Application Priority Data
Dec. 11, 2020 (IN) 202041053990
Dec. 19, 2020 (IN) 202041055352
Oct. 24, 2021 (IN) 202141048442

(51) Int. Cl. A61K 31/445; A61K 45/06

(52) U.S. Cl. CPC A61K 31/445; A61K 45/06

(57) ABSTRACT

The present disclosure provides a liquid formulation meant for nasal administration that includes fexofenadine or a salt or a hydrate or a solvate thereof. The compositions of the present disclosure may find utility in treatment of allergic conditions/diseases. The liquid formulation of the present disclosure gets converted into a gel upon its administration in the nasal region and releases the active agent locally in a sustained manner, while providing moisturizing effect and forming a layer on the nasal mucosa that acts like a barrier to entry of pathogens and allergens inhibiting their invasion into sinuses and deep layers of lining of the nose. Aspects of the present disclosure also relates to method of treating an allergic condition using the advantageous compositions of the present disclosure.

### FIELD OF THE INVENTION

The present disclosure generally relates to the field of pharmaceutical compositions. In particular, the present disclosure provides a liquid formulation meant for nasal administration that includes fexofenadine or a salt or a hydrate or a solvate thereof. The compositions of the present disclosure may find utility in treatment of allergic conditions/diseases.

### BACKGROUND OF THE INVENTION

Allergic diseases encompass a spectrum of disorders characterized by the development of an overactive immune response, characterized by excessive and inappropriate responses to allergens leading to a Th2 skewed immune environment. Multiple immune cells and inflammatory mediators contribute to the initiation and manifestation of allergic diseases. Allergic diseases/disorders are caused by allergen-induced unfavorable immune responses initiating various symptoms in different organs, which often cannot be completely controlled by modern medicine. Allergic diseases occur worldwide and their prevalence appears to have been on the increase in the last two decades.

Allergic diseases, including allergic rhinitis, food allergy, sinusitis, hay fever, asthma etc. are the most common immunological diseases affecting children, and the prevalence of these conditions has risen in recent years. At a cellular level, it results from the interaction of allergen with allergen specific IgE on the surface of mast cells and basophils, resulting in the release in chemical mediators and the influx of inflammatory cells. Allergic diseases can deeply interfere with patient's quality of life, with detrimental effects on the physical, psychological, and social dimensions of life.

Allergen binding to IgE receptors on mast cells initiates a cascade of signaling events leading to the production of potent inflammatory mediators including histamine, platelet-activating factor, IL-6 and many others. Immunoglobulin E (IgE) antibodies and mast cells contribute substantially to disease development, progression and organ-localized pathology in many people afflicted with asthma and other allergic disorders.

The role of histamine in the pathophysiology of allergic disorders has been well-recognized. Histamine, which is stored mainly in mast cells and basophils, is a prominent contributor to allergic disease. Elevations in plasma or tissue histamine levels have been noted during anaphylaxis and experimental allergic responses of the skin, nose, and airways. In allergic rhinitis, histamine is capable of mediating the pruritus, mucosal edema and sneezing through its H1 receptors. In the nose, mucus secretion can be reflexively mediated by H1 receptors. Histamine plays a primary role in allergic diseases.

Blockade of histamine H1 receptor, the anti-inflammatory effects have increasingly been recognized to play an important role in the management of allergic diseases, rhinitis, asthma, and urticaria. H1 antihistamine agent such as fexofenadine play a pivotal role in the treatment of allergic diseases and are among the most prescribed medications in the world. Depending on their action on the central nervous system, H1 antihistamines are classified as first-generation and second-generation. Most first-generation H1-antihistamines have anticholinergic, sedative, local anesthetic, and anti-5-HT effects, which might favorably affect the symptoms of the allergic response but also contribute to side-effects. It is clear that, H1-antihistamines have multiple effects on the allergic inflammatory response.

The mechanisms underlying chronic allergy is highly complex and involve multiple immune cells, mediators, and cytokines. Thus the development of a single drug to treat allergic inflammation and/or symptoms is disconcerting by the complexity of the disease pathophysiology. Current available therapeutic options are typically focused on achieving symptomatic relief.

Nonsedating antihistamines are recommended as first-line treatment for patients with urticaria. In chronic spontaneous urticaria, wheals arise spontaneously for more than 6 weeks without external physical stimuli. Histamine is the main mediator of urticaria and H1-receptor antagonists represent the treatment of choice in all patients with chronic urticaria. However, the efficacy is minimal compared to other agents for the treatment of CSU.

Although numerous compositions have been reported so far, they suffer from one or more shortcomings. There is, therefore, a need for improved compositions and methods that can be used to treat a wide variety of allergic diseases/disorders especially, patients with severe conditions need to ameliorate the multiple symptoms in a short duration to improve the patients' quality of life. A need is also felt of improved formulations that are easy to administer and aids in improving patient compliance. The present disclosure satisfies the existing needs, at least in part, and overcomes one or more disadvantages of the conventional approaches.

### OBJECTS OF THE INVENTION

One of the objects of the present disclosure is to provide a pharmaceutical composition that may overcome the limitations associated with the conventional compositions.

Another object of the present disclosure is to provide a pharmaceutical composition that exhibits superior storage stability and functional reciprocity.

Further object of the present disclosure is to provide a pharmaceutical composition that is easy to prepare and is economical.

Yet another object of the present disclosure is to provide a pharmaceutical composition that finds utility in treatment for a wide variety of allergic diseases/conditions including severe/chronic allergic conditions.

One further object of the present invention is to ameliorate the multiple symptoms in a short duration to improve the patients' quality of life.

Still another object of the present disclosure is to deliver the active agents either simultaneously or concurrently or concomitantly to a subject for treatment of the disease.

Still further object of the present disclosure is to provide a pharmaceutical composition that turns into a gel upon administration in the nasal region and releases the active ingredients locally in a sustained manner.

### SUMMARY OF THE INVENTION

The present disclosure generally relates to the field of pharmaceutical compositions. In particular, the present disclosure provides a liquid formulation meant for nasal administration that includes fexofenadine or a salt or a hydrate or a solvate thereof. The compositions of the present disclosure may find utility in treatment of allergic conditions/diseases including server/chronic allergic conditions and to ameliorate the multiple symptoms in a short duration to improve the patients' quality of life.

An aspect of the present disclosure provides a pharmaceutical composition including: fexofenadine or a salt or a hydrate or a solvate thereof, said composition being formulated as a liquid formulation meant for nasal administration. In an embodiment, the liquid formulation turns into a gel upon nasal administration. In an embodiment, the composition is formulated as a nasal spray.

In an embodiment, the pharmaceutical composition comprises: fexofenadine or a salt or a hydrate or a solvate thereof, and an in-situ gel forming polymer. Inclusion of in-situ gel forming polymer as part of the composition may aid in conversion of the composition into a gel upon administration in the nasal region.

Another aspect of the present disclosure provides a pharmaceutical composition including: fexofenadine or a salt or a hydrate or a solvate thereof and a zinc salt, said composition being formulated as a liquid formulation meant for nasal administration. In an embodiment, the composition is formulated as a nasal spray.

The composition of the present disclosure includes fexofenadine or a salt or a hydrate or a solvate thereof and a zinc salt in a weight ratio ranging from 1:1 to 1:20. In an embodiment, fexofenadine or a salt or a hydrate or a solvate thereof and a zinc salt are present in the composition in a weight ratio ranging from 1:1.5 to 1:10.

In an embodiment, the composition also includes an excipient. The excipient is selected from any or a combination of: a diluent, an anti-oxidant, a preservative, a solvent, a polyhydric alcohol, a sugar alcohol, a fatty acid or derivative thereof, an amino acid or metabolite or derivative thereof, a surfactant, a solubilizer and a stabilizer.

In accordance with an embodiment of the present disclosure, the composition comprises: fexofenadine or a salt or a hydrate or a solvate thereof in an amount ranging from 0.02% w/v to 2% w/v; a zinc salt in an amount ranging from 0.02% w/v to 5% w/v; a polyhydric alcohol in an amount ranging from 5% w/v to 30% w/v; a surfactant in an amount ranging from 1% w/v to 30% w/v; a sugar alcohol in an amount ranging from 0.5% w/v to 25% w/v; and water in an amount ranging from 35% w/v to 90% w/v.

In an embodiment, the polyhydric alcohol is selected from polyhydric alkanes, polyhydric alkane esters, polyalkene glycols, and mixtures thereof. In an embodiment, the sugar alcohol is selected from sorbitol, xylitol, mannitol, maltitol, inositol, allitol, altriol, dulcitol, galactitol, glucitol, hexitol, iditol, pentitol, ribitol, erythritol, and mixtures thereof. In an embodiment, the composition further includes a preservative benzalkonium chloride, benzyl alcohol and potassium sorbate in an amount ranging from 0.00001% w/v to 5% w/v, said preservative being benzyl alcohol. In an embodiment, the surfactant comprises a combination of microcrystalline cellulose and sodium carboxymethyl cellulose.

In some embodiments, the pharmaceutical composition comprises: fexofenadine or a salt or a hydrate or a solvate thereof in combination with any or a combination of: a corticosteroid, an anti-histamine, an anti-allergy agent, an aminothiol, an anti-inflammatory agent, an immunosuppressants, an NO (nitric oxide) releasing drugs, an PDE inhibitors, 5-HT1 agonists, ergolines, TCAs, anticonvulsant, an JAK Inhibitor and a bronchodilator. The pharmaceutical composition also includes at least one excipient and a carrier.

The advantageous liquid formulations of the present disclosure convert into a gel upon its administration in the nasal region and releases fexofenadine locally in a sustained manner, while providing moisturizing effect. The advantageous liquid formulations of the present disclosure also form a layer on the nasal mucosa, which acts like a barrier to entry of pathogens and allergens inhibiting their invasion into sinuses and deep layers of lining of the nose. Accordingly, the formulations of the present disclosure can find utility in treatment of a wide variety of allergic conditions/diseases/disorders.

In an embodiment, present invention provide a method for manufacturing the nasal pharmaceutical composition. The method comprises (a) preparing a premix 1, wherein the premix 1, comprises of adding ZnSO4.7H2O, xylitol, glycerol, and water, in a compounding vessel to obtain a mixture using an overhead stirrer; (b) preparing a premix 2, wherein in premix 2, comprises of propylene glycol, benzyl alcohol and polysorbate 20 or polysorbate 80 or one or more other excipients is selected and mixed in another compounding vessel using magnetic stirrer; (c) fexofenadine HCl is added to the premix 2 and allowed to dissolve completely; (d) solution from step (c) is mixed into premix 1 from step (a) and allowed to dissolve completely to obtain the desired composition; and the final volume of the nasal composition of step (d) is adjusted with water. In certain embodiment, for manufacturing the nasal composition by including one or more additional pharmaceutical agents with fexofenadine HCl can be prepared by adding the additional pharmaceutical active agent with fexofenadine HCl to the premix 2 in step (c). Further the steps (a) and (b), optional include one or more additional solvents or one or more other excipients.

### DETAILED DESCRIPTION

The present disclosure generally relates to the field of pharmaceutical compositions.

As used herein, the following terms and phrases shall have the meanings set forth below. Unless defined otherwise, all technical and scientific terms used herein have the same meaning as commonly understood to one of ordinary skill in the art. It is also understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, “an active agent” or “an active ingredient” refers not only to a single active agent but also to a combination of two or more different active agents, “a dosage form” refers to a combination of dosage forms as well as to a single dosage form, and the like.

The term “active agent” or “therapeutic agent”, encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds.

The term “combination therapy” or “combined treatment” or “in combination” as used herein denotes any form of concurrent or concomitantly or co-administration of active agents for treating acute and chronic allergic conditions such as allergic rhinitis, food allergy, sinusitis, hay fever, asthma, upper respiratory infections, nasal allergy, chronic rhinosinusitis, common cold but not limited thereto, which may be triggered upon a subject coming into contact with allergen(s).

The terms “treating” and “treatment” as used herein refers to reduction in severity and/or frequency of symptoms, elimination of symptoms and/or underlying cause, and improvement or remediation of damage caused thereby. Thus, “treating” a subject/patient as described herein encompasses treating a wide variety of allergic conditions such as allergic rhinitis, food allergy, sinusitis, hay fever, asthma, but not limited thereto, which may be triggered upon a subject coming into contact with allergen(s).

The term “dosage form” denotes any form of a pharmaceutical composition that contains an amount of active agent sufficient to elicit a desired therapeutic response.

The term “controlled release” refers to a drug-containing formulation or fraction thereof in which release of the drug is not immediate. The term “controlled release” as used herein includes sustained release, non-immediate release and delayed release formulations.

The term “sustained release” (synonymous with “extended release”) is used in its conventional sense to refer to a drug formulation that provides for gradual release of a drug over an extended period of time.

The term “pharmaceutically acceptable” means the material incorporated into a pharmaceutical composition that can be administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

“Pharmacologically active” (or simply “active”) as in a pharmacologically active derivative or analog, refers to a derivative or analog having the same type of pharmacological activity as the parent compound and approximately equivalent in degree.

The pharmaceutical composition provided in the present disclosure includes an anti-inflammatory agents such as antihistamines to suppress the distribution of certain mediators seen in the allergic conditions. The H1 receptor provides a positive regulation of a molecular transcription factor, NF-κB. Antihistamines can stabilise the inactive form of the receptor and for this reason possess receptor-dependent, anti-inflammatory characteristics. The H1 antihistamines (fexofenadine hydrochloride) of the present invention reduce the sedating side effects (low plasma and tissue peak levels) resulting in improved risk-benefit relationship and pharmacokinetics. The fexofenadine hydrochloride is a second-generation, selective histamine H1-receptor antagonist.

An aspect of the present disclosure provides a pharmaceutical composition including: fexofenadine or a salt or a hydrate or a solvate thereof, said composition being formulated as a liquid formulation meant for nasal administration. In an embodiment, the liquid formulation turns into a gel upon nasal administration. In an embodiment, the composition is formulated as a nasal spray.

In an embodiment, the pharmaceutical composition comprises: fexofenadine or a salt or a hydrate or a solvate thereof and an in-situ gel forming polymer. Inclusion of in-situ gel forming polymer as part of the composition affords conversion of the composition into a gel upon administration in the nasal region. Non-limiting examples of in situ gel forming polymers include mucoadhesive polymers and thermosensitive polymers. The non-limiting examples of such polymers include carbopol 934P, chitosan, sodium carboxymethyl cellulose (NaCMC), hydroxypropyl methylcellulose (HPMC), hydroxypropyl cellulose, poly(acrylic acid), pluronic, poloxamer gel, poloxamer F127, N-trimethyl chitosan chloride, N-[(2-hydroxy-3-trimethylammonium)propyl] chitosan chloride (HTCC), Chitosan-polyvinyl alcohol, Poly(N-isopropylacrylamide) (PNiPAAm), polymethacrylic acid, polyethylene glycol, polyveinylacetal diethylamino acetate, gellan gum, alginic acid, xyloglucan, pectin, chitosan, poly(DL-lactic acid), poly(DL-lactide-co-glycolide), poly-caprolactone, Poly(ether-ester) based biodegradable block copolymers such as poly(ethylene oxide)-poly(lactic acid) (PEO-PLA) copolymer, poly(ethyleneoxide)-poly(caprolactone) (PEO-PCL), poly(ethyleneglycol)-poly(lactide-co-glycolide)-poly(ethylene glycol) (PEG-PLGA-PEG) and combinations thereof.

In one embodiment the pharmaceutical compositions and formulations of the present disclosure comprising a fixed dose combination of fexofenadine HCL, xylitol, zinc sulphate, propylene glycol, polysobate 80, glycerin, VIVAPUR MCG811, water with other excipients (composition 1) is beneficial in the suppressing of symptoms associated with allergic conditions and is also potent in acuter upper respiratory infections.

In some embodiments, the pharmaceutical composition further includes any or a combination of: corticosteroid, 5-HT1 agonists, ergolines, TCAs, anticonvulsant, anti-histamine, aminothiol, anti-allergy agent, anti-inflammatory agent, Immunosuppressant, NO(nitric oxide) releasing drugs, phosphodiesterase (PDE) inhibitors, Janus kinase (JAK) Inhibitor, 5-HT1 agnostic, TCAs, and bronchodilator in effective therapeutic amounts for the treatment of the particular disease or condition.

### Further Embodiments

Composition 1a: Composition 1 in combination with corticosteroid.

Composition 1b: Composition 1 in combination other antihistamine.

Composition 1c: Composition 1 in combination with aminothiol.

Composition 1d: Composition 1 in combination with anti-allergy agent.

Composition 1e: Composition 1 in combination with anti-inflammatory agent.

Composition 1f: Composition 1 in combination with Immunosuppressant.

Composition 1g: Composition 1 in combination with NO releasing drugs.

Composition 1h: Composition 1 in combination with PDE inhibitors.

Composition 1i: Composition 1 in combination with JAK Inhibitor.

Composition 1j: Composition 1 in combination with bronchodilator.

Composition 1ja: Composition 1 in combination with anticonvulsant.

Composition 1jb: Composition 1 in combination with ergolines.

Composition 1jc: Composition 1 in combination with 5-HT1 agonists.

Composition 1jd: Composition 1 in combination with TCAs.

Composition 1k: Composition 1a in combination with aminothiol.

Composition 1l: Composition 1a in combination with anti-allergy agent.

Composition 1m: Composition 1a in combination with anti-inflammatory agent.

Composition 1n: Composition 1a in combination with Immunosuppressant.

Composition 1o: Composition 1a in combination with NO releasing drugs.

Composition 1p: Composition 1a in combination with PDE inhibitors.

Composition 1q: Composition 1a in combination with JAK Inhibitor.

Composition 1r: Composition 1a in combination with bronchodilator.

Composition 1pa: Composition 1a in combination with anticonvulsant.

Composition 1qb: Composition 1a in combination with ergolines.

Composition 1rc Composition 1a in combination with 5-HT1 agonists.

Composition 1sd: Composition 1c in combination with TCAs.

Composition 1s: Composition 1c in combination with corticosteroid.

Composition 1t: Composition 1c in combination with anti-allergy agent.

Composition 1u: Composition 1c in combination with anti-inflammatory agent.

Composition 1v: Composition 1c in combination with Immunosuppressant.

Composition 1w: Composition 1c in combination with NO releasing drugs.

Composition 1x: Composition 1c in combination with PDE inhibitors.

Composition 1y: Composition 1c in combination with JAK Inhibitor.

Composition 1z: Composition 1c in combination with bronchodilator.

Composition 1wa: Composition 1c in combination with anticonvulsant.

Composition 1xb: Composition 1c in combination with ergolines.

Composition 1yc: Composition 1c in combination with 5-HT1 agonists.

Composition 1zd: Composition 1c in combination with TCAs.

Composition 2a: Composition 1d in combination with aminothiol.

Composition 2b: Composition 1d in combination with corticosteroid.

Composition 2c: Composition 1d in combination with anti-inflammatory agent.

Composition 2d: Composition 1d in combination with Immunosuppressant.

Composition 2e: Composition 1d in combination with NO releasing drugs.

Composition 2f: Composition 1d in combination with PDE inhibitors.

Composition 2g: Composition 1d in combination with JAK Inhibitor.

Composition 2h: Composition 1d in combination with bronchodilator.

Composition 2ea: Composition 1d in combination with anticonvulsant.

Composition 2fb: Composition 1d in combination with ergolines.

Composition 2gc: Composition 1d in combination with 5-HT1 agonists.

Composition 2hd: Composition 1d in combination with TCAs.

Composition 2i: Composition 1e in combination with aminothiol.

Composition 2j: Composition 1e in combination with corticosteroid.

Composition 2k: Composition 1e in combination with anti-allergic agent.

Composition 2l: Composition 1e in combination with Immunosuppressant.

Composition 2m: Composition 1e in combination with NO releasing drugs.

Composition 2n: Composition 1e in combination with PDE inhibitors.

Composition 2o: Composition 1e in combination with JAK Inhibitor.

Composition 2p: Composition 1e in combination with bronchodilator.

Composition 2ma: Composition 1e in combination with anticonvulsant.

Composition 2nb: Composition 1e in combination with ergolines.

Composition 2oc: Composition 1e in combination with 5-HT1 agonists.

Composition 2pd: Composition 1e in combination with TCAs.

Composition 2q: Composition 1f in combination with aminothiol.

Composition 2r: Composition 1f in combination with corticosteroid.

Composition 2s: Composition 1f in combination with anti-allergic agent.

Composition 2t: Composition 1f in combination with anti-inflammatory agent.

Composition 2u: Composition 1f in combination with NO releasing drugs.

Composition 2v: Composition 1f in combination with PDE inhibitors.

Composition 2w: Composition 1f in combination with JAK Inhibitor.

Composition 2x: Composition 1f in combination with bronchodilator.

Composition 2ua: Composition 1f in combination with anticonvulsant.

Composition 2vb: Composition 1f in combination with ergolines.

Composition 2wc: Composition 1f in combination with 5-HT1 agonists.

Composition 2xd: Composition 1f in combination with TCAs.

Composition 2y: Composition 1g in combination with aminothiol.

Composition 2z: Composition 1g in combination with corticosteroid.

Composition 3a: Composition 1g in combination with anti-allergic agent.

Composition 3b: Composition 1g in combination with anti-inflammatory agent.

Composition 3c: Composition 1g in combination with Immunosuppressant.

Composition 3d: Composition 1g in combination with PDE inhibitors.

Composition 3e: Composition 1g in combination with JAK Inhibitor.

Composition 3f: Composition 1g in combination with bronchodilator.

Composition 3g: Composition 1h in combination with aminothiol.

Composition 3h: Composition 1h in combination with corticosteroid.

Composition 3i: Composition 1h in combination with anti-allergic agent.

Composition 3j: Composition 1h in combination with anti-inflammatory agent.

Composition 3k: Composition 1h in combination with Immunosuppressant.

Composition 3l: Composition 1h in combination with NO releasing drugs.

Composition 3m: Composition 1h in combination with JAK Inhibitor.

Composition 3n: Composition 1h in combination with bronchodilator.

Composition 3o: Composition 1i in combination with aminothiol.

Composition 3p: Composition 1i in combination with corticosteroid.

Composition 3q: Composition 1i in combination with anti-allergic agent.

Composition 3r: Composition 1i in combination with anti-inflammatory agent.

Composition 3s: Composition 1i in combination with Immunosuppressant.

Composition 3t: Composition 1i in combination with NO releasing drugs.

Composition 3u: Composition 1i in combination with PDE inhibitors.

Composition 3v: Composition 1i in combination with bronchodilator.

Composition 3w Composition 1 in combination with 5-HT1 agonists.

Composition 3x: Composition 1 in combination with TCAs.

Composition 3y: Composition 1 in combination with anticonvulsant.

Composition 3z: Composition 1a in combination with ergolines.

Composition 4a: Composition 1a to 3v 1z in combination with 5-HT1 agonists.

Composition 4b: Composition 1a to 3v in combination with TCAs.

Composition 4c: Composition 1a to 3v in combination with anticonvulsant.

Composition 4d: Composition 1a to 3v in combination with ergolines.

In some embodiments, non-limiting examples of corticosteroids, anti-histamines, anti-allergy, aminothiol, anti-inflammatory agents and bronchodilators include: beclomethasone, budesonide, ciclesonide, flunisolide, fluticasone furoate, fluticasone propionate, mometasone, triamcinolone, prednisone, desloratadine, azelastine, cetirizine, terfenadine, chlorphenamine, levocetirizine, montelukast, loratadine, bilastine, levalbuterol, olopatadine, brompheniramine, benralizumab, chlorpheniramine, clemastine, cromolyn, cyproheptadine, ibuprofen, diphenhydramine, hydroxyzine, promethazine, triprolidine, ketotifen, naphazoline, pheniramine, methylprednisolone, dexamethasone, pseudoephedrine, phenylephrine, Albuterol, levalbuterol, ipratropium bromide, vilanterol, salbutamol, salmeterol, formoterol, oxymetazoline, xylometazoline, amidrine, beclomethasone, ciclesonide, fluticasone furoate, mometasone, budesonide, fluticasone, triamcinolone, glycopyrronium, tiotropium, arformoterol, theophylline, aminophylline, ipratropium, bitolterol, carbuterol, fenoterol, isoetarine, pirbuterol, procaterol, reproterol, rimiterol, salbutamol, levosalbutamol, terbutaline, tulobuterol, bambuterol, clenbuterol, formoterol/arformoterol, salmeterol, salmefamol, abediterol, carmoterol, indacaterol, olodaterol, vilanterol, epinephrine, hexoprenaline, isoprenaline (isoproterenol), orciprenaline (metaproterenol), beclomethasone, budesonide, ciclesonide, flunisolide, fluticasone propionate, mometasone, triamcinolone, aclidinium bromide, ipratropium bromide, oxitropium bromide, tiotropium bromide, umeclidinium bromide, acefylline, ambuphylline, aminophylline, bamifylline, choline theophyllinate, caffeine, doxofylline, enprofylline, etamiphylline, proxyphylline, theophylline, montelukast, pranlukast, zafirlukast, zileuton, ramatroban, seratrodast, cysteamine HCl and combinations thereof.

In some embodiments, non-limiting examples of immunosuppressant includes azathioprine, mycophenolic acid, leflunomide, teriflunomide, ciclosporin, pimecrolimus, tacrolimus, voclosporin, lenalidomide, pomalidomide, thalidomide, apremilast, sirolimus, everolimus, ridaforolimus, temsirolimus, umirolimus, zotarolimus, baricitinib, blisibimod, nilotinib, filgotinib, tofacitinib, upadacitinib, abatacept, belatacept, etanercept pegsunercept, aflibercept alefacept rilonacept and combinations thereof.

In some embodiments, non-limiting examples of NO releasing drugs include glyceryl trinitrate, isosorbide dinitrate, isosorbide mononitrate, isoamyl nitrite and other derivatives and analogs with the NO releasing properties.

In some embodiments, non-limiting examples of 5-HT1 agonists include almotriptan, avitriptan, eletriptan, frovatriptan, naratriptan, rizatriptan, sumatriptan, zolmitriptan and other derivatives and analogs and combinations thereof.

In some embodiments, non-limiting examples of ergolines include dihydroergocryptine, dihydroergotamine, ergotamine, lisuride methylergometrine, methysergide and other derivatives and analogs and combinations thereof.

In some embodiments, non-limiting examples of TCAs include amitriptyline, nortriptyline, imipramine, and other derivatives and analogs and combinations thereof.

In some embodiments, non-limiting examples of anticonvulsant include carbamazepine, oxcarbazepine, topiramate, valproate and other derivatives and analogs and combinations thereof.

In some embodiments, non-limiting examples of PDE 4 inhibitor include apremilast, arofylline, atizoram, benafentrine, catramilast, CC-1088, CDP-840, CGH-2466, cilomilast, cipamfylline, crisaborole, denbutylline, difamilast, drotaverine, etazolate, filaminast, glaucine, HT-0712, ICI-63197 indimilast, irsogladine, lavamilast, lirimilast, lotamilast, luteolin, mesembrenone, mesembrine, mesopram, oglemilast, piclamilast, pumafentrine, revamilast, Ro 20-1724, roflumilast, rolipram, ronomilast, RPL-554, RS-25344, tetomilast, tofimilast, YM-976, zardaverine, ibudilast, roflumilast and combination thereof.

In some embodiments, non-limiting examples of PDE 3 inhibitor include adibendan, amrinone (inamrinone), anagrelide, benafentrine, bucladesine, carbazeran, cilostamide, cilostazol, enoximone, imazodan, KMUP-1, meribendan, milrinone, olprinone, parogrelil, pimobendan, pumafentrine, quazinone, RPL-554, siguazodan, trequinsin, vesnarinone, zardaverine and combination thereof.

In some embodiments, non-limiting examples of PDE 5 inhibitor include acetildenafil, aildenafil, avanafil, beminafil, benzamidenafil, dasantafil, icariin, gisadenafil, homosildenafil, lodenafil, mirodenafil, MY-5445, nitrosoprodenafil, norcarbodenafil, SCH-51866, sildenafil, sulfoaildenafil, T-0156, tadalafil, udenafil, vardenafil and combination thereof.

In some embodiments, non-limiting examples of JAK Inhibitor include abrocitinib, baricitinib, filgotinib, momelotinib, oclacitinib, peficitinib, ruxolitinib, tofacitinib, tasocitinib, CP-690550, upadacitinib, atiprimod, AZD-1480, baricitinib, chz868, cucurbitacin I (elatericin B, JSI-124) CYT387 lestaurtinib, NSC-7908, NSC-33994, pacritinib, peficitinib, ruxolitinib, SD-1008, cercosporamide, decernotinib (VX-509), peficitinib, TCS-21311, WHI-P 15 ZM-39923, ZM-449829 and combination thereof.

Exemplified fixed dose combination of the compositions 1, 1a to 1z, 2a to 2z and 3a to 3v are as disclosed below. This embodiment are only an examples for understanding the invention.

Composition 1 as a non-limiting example:

| | Quality | | | |
|---|---|---|---|---|
| Ingredients | Reference | Function | mg/ml | % w/v |
| Fexofenadine. HCL | USP | API | 0.5 | 0.05 |
| Zinc sulphate | USP | Antimicrobial | 1.2 | 0.12 |
| (Heptahydrate) | | agent | | |
| Xylitol powder | USP | Antimicrobial | 20 | 2.0 |
| | | agent | | |
| Propylene glycol | USP | Solvent | 50 | 5.0 |
| Polysorbate 80 | USP | Surfactant | 0.1 | 0.01 |
| Glycerin | USP | Humectant | 50 | 5.0 |
| MCC/NaCMC | USP | Mucoadhesive | 20 | 2.0 |
| (VIVAPUR MCG 811) | | polymer | | |
| Purified Water | Millipore | Diluent | QS to | QS to |
| | | | 1 ml | 100 |

In one embodiment the exemplified fixed dose combination comprises of fexofenadine HCl, xylitol, zinc sulphate, fluticasone, propylene glycol, polysorbate 80, glycerin, VIVAPUR MCG811 with other excipients. Fluticasone in this composition may be replaced with any other known corticosteroid.

In one embodiment the exemplified fixed dose combination comprises of fexofenadine HCl, xylitol, zinc sulphate, levocetirizine, propylene glycol, polysorbate 80, glycerin, VIVAPUR MCG811 with other excipients. Levocetirizine in this composition may be replaced with any other known anti-histamine.

In another embodiment the exemplified fixed dose combination comprises of fexofenadine HCL, xylitol, zinc sulphate, cysteamine HCl, propylene glycol, polysorbate 80, glycerin, VIVAPUR MCG811 with other excipients. Cysteamine HCl in this composition may be replaced with any other known aminothiol.

In another embodiment the exemplified fixed dose combination comprises of fexofenadine HCL, xylitol, zinc sulphate, diphenhydramine, propylene glycol, polysorbate 80, glycerin, VIVAPUR MCG811 with other excipients. Diphenhydramine in this composition may be replaced with any other known anti-allergic agents.

In another embodiment the exemplified fixed dose combination comprises of fexofenadine HCL, xylitol, zinc sulphate, ibuprofen, propylene glycol, polysorbate 80, glycerin, VIVAPUR MCG811 with other excipients. Ibuprofen in this composition may be replaced with any other known anti-inflammatory agents.

In another embodiment the exemplified fixed dose combination comprises of fexofenadine HCL, xylitol, zinc sulphate, leflunomide, propylene glycol, polysorbate 80, glycerin, VIVAPUR MCG811 with other excipients. Leflunomide in this composition may be replaced with any other known immunosuppressant.

In another embodiment the exemplified fixed dose combination comprises of fexofenadine HCL, xylitol, zinc sulphate, glyceryl trinitrate, propylene glycol, polysorbate 80, glycerin, VIVAPUR MCG811 with other excipients. Glyceryl trinitrate in this composition may be replaced with any other known NO releasing agents.

In another embodiment the exemplified fixed dose combination comprises of fexofenadine HCL, xylitol, zinc sulphate, apremilast, propylene glycol, polysorbate 80, glycerin, VIVAPUR MCG811 with other excipients. Apremilast, in this composition may be replaced with any other known PDE 4 inhibitors.

In another embodiment the exemplified fixed dose combination comprises of fexofenadine HCL, xylitol, zinc sulphate, adibendan, propylene glycol, polysorbate 80, glycerin, VIVAPUR MCG811 with other excipients. Adibendan in this composition may be replaced with any other known PDE 3 inhibitors.

In another embodiment the exemplified fixed dose combination comprises of fexofenadine HCL, xylitol, zinc sulphate, acetildenafil, propylene glycol, polysorbate 80, glycerin, VIVAPUR MCG811 with other excipients. Acetildenafil in this composition may be replaced with any other known PDE5 inhibitors.

In another embodiment the exemplified fixed dose combination comprises of fexofenadine HCL, xylitol, zinc sulphate, abrocitinib propylene glycol, polysorbate 80, glycerin, VIVAPUR MCG811 with other excipients. Abrocitinib in this composition may be replaced with any other known JAK inhibitors.

In an embodiment the exemplified fixed dose combination comprises of fexofenadine HCL, xylitol, zinc sulphate, salmeterol, propylene glycol, polysorbate 80, glycerin, VIVAPUR MCG811 with other excipients. Salmeterol may be in this composition replaced by any other bronchodilator.

In one embodiment the exemplified fixed dose combination comprises of fexofenadine HCL, xylitol, zinc sulphate, fluticasone, cysteamine HCl, propylene glycol, polysorbate 80, glycerin, VIVAPUR MCG811 with other excipients. Fluticasone and cysteamine HCL in this composition may be replaced with any other known corticosteroid or aminothiol.

In another embodiment the exemplified fixed dose combination comprises of fexofenadine HCL, xylitol, zinc sulphate, fluticasone, diphenhydramine, propylene glycol, polysorbate 80, glycerin, VIVAPUR MCG811 with other excipients. Diphenhydramine and fluticasone in this composition may be replaced with any other known anti-allergic agents or corticosteroid.

In another embodiment the exemplified fixed dose combination comprises of fexofenadine HCL, xylitol, zinc sulphate, fluticasone, ibuprofen, propylene glycol, polysorbate 80, glycerin, VIVAPUR MCG811 with other excipients. Ibuprofen and fluticasone in this composition may be replaced with any other known anti-inflammatory agents or corticosteroid.

In another embodiment the exemplified fixed dose combination comprises of fexofenadine HCL, xylitol, zinc sulphate, fluticasone, leflunomide, propylene glycol, polysorbate 80, glycerin, VIVAPUR MCG811 with other excipients. Leflunomide and fluticasone in this composition may be replaced with any other known immunosuppressants or corticosteroid.

In another embodiment the exemplified fixed dose combination comprises of fexofenadine HCL, xylitol, zinc sulphate, fluticasone, glyceryl trinitrate, propylene glycol, polysorbate 80, glycerin, VIVAPUR MCG811 with other excipients. Glyceryl trinitrate and fluticasone in this composition may be replaced with any other known NO releasing agents or corticosteroid.

In another embodiment the exemplified fixed dose combination comprises of fexofenadine HCL, xylitol, zinc sulphate, fluticasone, apremilast, propylene glycol, polysorbate 80, glycerin, VIVAPUR MCG811 with other excipients. Apremilast and fluticasone in this composition may be replaced with any other known PDE 4 inhibitors or corticosteroid.

In another embodiment the exemplified fixed dose combination comprises of fexofenadine HCL, xylitol, zinc sulphate, fluticasone, adibendan, propylene glycol, polysorbate 80, glycerin, VIVAPUR MCG811 with other excipients. Adibendan and fluticasone in this composition may be replaced with any other known PDE 3 inhibitors or corticosteroid.

In another embodiment the exemplified fixed dose combination comprises of fexofenadine HCL, xylitol, zinc sulphate, fluticasone, acetildenafil, propylene glycol, polysorbate 80, glycerin, VIVAPUR MCG811 with other excipients. Acetildenafil and fluticasone in this composition may be replaced with any other known PDE5 inhibitors or corticosteroid.

In another embodiment the exemplified fixed dose combination comprises of fexofenadine HCL, xylitol, zinc sulphate, fluticasone, abrocitinib propylene glycol, polysorbate 80, glycerin, VIVAPUR MCG811 with other excipients. Abrocitinib and fluticasone in this composition may be replaced with any other known JAK inhibitors or corticosteroid.

In an embodiment the exemplified fixed dose combination comprises of fexofenadine HCL, xylitol, zinc sulphate, salmeterol, fluticasone, propylene glycol, polysorbate 80, glycerin, VIVAPUR MCG811 with other excipients. Salmeterol and fluticasone may be in this composition replaced by any other bronchodilator or corticosteroid.

In another embodiment the exemplified fixed dose combination comprises of fexofenadine HCL, xylitol, zinc sulphate, cysteamine HCL, fluticasone, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, Diphenhydramine, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, ibuprofen, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, leflunomide, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, glyceryl trinitrate, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, apremilast, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, adibendan, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, acetildenafil, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, abrocitinib, propylene glycol, polysorbate 80, glycerin, VIVAPUR MCG811 with other excipients.

In another embodiment the exemplified fixed dose combination comprises of fexofenadine HCL, xylitol, zinc sulphate, cysteamine HCL, salmeterol, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, fluticasone, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, ibuprofen, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, leflunomide, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, glyceryl trinitrate, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, apremilast, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, adibendan, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, acetildenafil, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, abrocitinib, propylene glycol, polysorbate 80, glycerin, VIVAPUR MCG811 with other excipients.

In another embodiment the exemplified fixed dose combination comprises of fexofenadine HCL, xylitol, zinc sulphate, diphenhydramine, salmeterol, propylene glycol, polysorbate 80, glycerin, VIVAPUR MCG811 with other excipients.

In another embodiment the exemplified fixed dose combination comprises of fexofenadine HCL, xylitol, zinc sulphate, almotriptan propylene glycol, polysorbate 80, glycerin, VIVAPUR MCG811 with other excipients.

In another embodiment the exemplified fixed dose combination comprises of fexofenadine HCL, xylitol, zinc sulphate, dihydroergocryptine, propylene glycol, polysorbate 80, glycerin, VIVAPUR MCG811 with other excipients.

In another embodiment the exemplified fixed dose combination comprises of fexofenadine HCL, xylitol, zinc sulphate, amitriptyline, propylene glycol, polysorbate 80, glycerin, VIVAPUR MCG811 with other excipients.

In another embodiment the exemplified fixed dose combination comprises of fexofenadine HCL, xylitol, zinc sulphate, carbamazepine, propylene glycol, polysorbate 80, glycerin, VIVAPUR MCG811 with other excipients.

The present disclosure is also, in part, on the premise of surprising observation of the inventors of the present disclosure that the compositions and formulations of the present disclosure can afford a unique treatment option for allergic conditions/disorders, wherein fexofenadine being a H1 Histamine receptor blocker, affords treatment of allergic conditions/disorders, while zinc salts acts like a micronutrient which also provides antioxidant effects along with anti-microbial effects forming a protective layer on the mucosa while aiding in maintaining many biochemical and physiological processes at the molecular, cellular, and multiple organ and systemic levels, wherein alteration of zinc homeostasis may cause dysfunction of many organs and systems; further, zinc ions may prevent viruses such as rhinovirus from attaching to and infecting cells in the nasal cavity as well as Covid-19. It further improves the immune system reduces oxidative stress, prevent nuclear factor-kappa B (NF-kB)-DNA binding to mononuclear cells and also improves T-helper cell functions. Accordingly, the compositions of the present disclosure including fexofenadine and zinc salt exhibit strong functional reciprocity and synergy.

Accordingly, another aspect of the present disclosure provides a pharmaceutical composition comprising: fexofenadine or a salt or a hydrate or a solvate thereof and a zinc salt, said composition being formulated as a liquid formulation meant for nasal administration.

The composition of the present disclosure comprises fexofenadine or a salt or a hydrate or a solvate thereof and a zinc salt in a weight ratio ranging from 1:1 to 1:20. In an embodiment, the zinc salt is ZnSO4.7H2O.

Alternatively, the composition of the present disclosure comprises fexofenadine or a salt or a hydrate or a solvate thereof and a zinc salt in a weight ratio ranging from 1:1 to 1:15.

Alternatively, the composition of the present disclosure comprises fexofenadine or a salt or a hydrate or a solvate thereof and a zinc salt in a weight ratio ranging from 1:1.5 to 1:20.

Still alternatively, the composition of the present disclosure comprises fexofenadine or a salt or a hydrate or a solvate thereof and a zinc salt in a weight ratio ranging from 1:1.5 to 1:15.

Still alternatively, the composition of the present disclosure comprises fexofenadine or a salt or a hydrate or a solvate thereof and a zinc salt in a weight ratio ranging from 1:1.5 to 1:10.

In an embodiment, the pharmaceutical composition includes an osmolyte such as xylitol that may lower airway surface liquid salt concertation thus enhancing the activity of innate immune system. It may reduce or control the microbial load in the nasal cavity and prevent or slow the manifestation of the infection. Accordingly, the compositions of the present disclosure including fexofenadine and xylitol exhibit strong functional reciprocity and synergy.

In another embodiment, the pharmaceutical composition may include a moisture improving agent such as glycerin. This prevents dryness of nasal passage and pain associated.

In an embodiment, polysorbate 80 or polysorbate 20 or polysorbate as a non-ionic surfactant is used as a solvent, for enhancing the drug distribution and its absorption. Further use of propylene glycol helps in reducing the symptoms of obstruction and inflammation in the nose and VIVAPUR MCG 811 is used as emulsifying agent or suspending agent which also helps in the absorption of the drug.

In an embodiment, the composition includes an excipient. The excipient is selected from any or a combination of: a diluent, an anti-oxidant, a preservative, a solvent, a polyhydric alcohol, an isotonicity adjusting agent, a pH adjusting agent, a buffer, a co-solvent, a humectant, a sugar alcohol, an in-situ gel forming polymer, a fatty acid or derivative thereof, an amino acid or metabolite or derivative thereof, a surfactant, a solubilizer and a stabilizer.

The composition may be formulated as a semi-solid or liquid dosage form, preferably, in a liquid dosage form. Non-limiting examples of dosage forms includes suspension, solution, emulsion, powder, aerosol sprays, cream, ointment, lotion, gel and the likes. In an embodiment, the composition is formulated as a liquid formulation meant for nasal administration. In an embodiment, the composition is formulated as a nasal spray.

In accordance with an embodiment of the present disclosure, the composition includes: a zinc salt in an amount ranging from 0.02% w/v to 5% w/v; a polyhydric alcohol in an amount ranging from 5% w/v to 30% w/v; a surfactant in an amount ranging from 1% w/v to 30% w/v; a sugar alcohol in an amount ranging from 0.5% w/v to 25% w/v; water in an amount ranging from 35% w/v to 90% w/v. In an embodiment, the composition further includes a preservative in an amount ranging from 0.01% w/v to 5% w/v.

Examples of polyhydric alcohols useful in the compositions of the present disclosure include polyhydric alkanes, polyhydric alkane esters, polyalkene glycols, and mixtures thereof. Polyhydric alkanes can be propylene glycol, glycerin, glycerol, butylene glycol, hexylene glycol, 1, 3-propanediol and the likes, but not limited thereto. Polyhydric alkane esters can be dipropylene glycol, ethoxydiglycol and the likes, but not limited thereto. Polyalkene glycols can be polyethylene glycol, polypropylene glycol and the likes, but not limited thereto. The compositions of the present disclosure include polyhydric alcohol in an amount ranging from 5% w/v to 30% w/v, preferably, in an amount ranging from 5% w/v to 25% w/v, and more preferably, in an amount ranging from 5% w/v to 20% w/v.

Examples of antioxidants useful in the compositions of the present disclosure include sodium metabisulfite, vitamin A, tocopherol, ascorbic acid or salt or derivative thereof, tartaric acid or salt or derivative thereof, retinyl palmitate, sesamol, thiol derivatives, Butylated Hydroxy Anisole (BHA), Butylated Hydroxyl Toluene (BHT), and mixtures thereof. However, any other anti-oxidant(s), as known to or appreciated by a person skilled in the art can also be used to serve its/their intended purpose as laid down in embodiments of the present disclosure.

Examples of buffers useful in the compositions of the present disclosure include citric acid or salt or derivative thereof, benzoic acid or salt or derivative thereof, sorbic acid or salt or derivative thereof, succinic acid or salt or derivative thereof, a bicarbonate salt of alkali earth metal, amino acids, an acid salt of an amino acid, an alkali salt of an amino acid and mixtures thereof. However, any other buffer(s), as known to or appreciated by a person skilled in the art can also be used to serve its/their intended purpose as laid down in embodiments of the present disclosure. In an embodiment, the buffer is citric acid or salt or derivative thereof.

Examples of sugar alcohols useful in the compositions of the present disclosure include sorbitol, xylitol, mannitol, maltitol, inositol, allitol, altriol, dulcitol, galactitol, glucitol, hexitol, iditol, pentitol, ribitol, erythritol, and mixtures thereof.

Non-limiting examples of in situ gel forming polymers include mucoadhesive polymers and thermosensitive polymers such as carbopol 934P, chitosan, sodium carboxymethyl cellulose (NaCMC), hydroxypropyl methylcellulose (HPMC), hydroxypropyl cellulose, poly(acrylic acid), pluronic, poloxamer gel, poloxamer F127, N-trimethyl chitosan chloride, N-[(2-hydroxy-3-trimethylammonium)propyl] chitosan chloride (HTCC), Chitosan-polyvinyl alcohol, Poly(N-isopropylacrylamide) (PNiPAAm), polymethacrylic acid, polyethylene glycol, polyveinylacetal diethylamino acetate, gellan gum, alginic acid, xyloglucan, pectin, chitosan, poly(DL-lactic acid), poly(DL-lactide-co-glycolide), poly-caprolactone, Poly(ether-ester) based biodegradable block copolymers such as poly(ethylene oxide)-poly(lactic acid) (PEO-PLA) copolymer, poly(ethyleneoxide)-poly(caprolactone) (PEO-PCL), poly(ethyleneglycol)-poly(lactide-co-glycolide)-poly(ethylene glycol) (PEG-PLGA-PEG) and combinations thereof. In an embodiment, the in situ gel forming polymer includes a combination of microcrystalline cellulose and sodium carboxymethyl cellulose, such as Vivapur® MCG 811p.

Examples of surfactants useful in the compositions of the present disclosure include anionic surfactants, nonionic surfactants, amphoteric surfactants and mixtures thereof. Anionic surfactants useful herein include, but are not limited to, sarcosine type surfactants or sarcosinates; taurates such as sodium methyl cocoyl taurate; alkyl sulfates such as sodium trideceth sulfate or sodium lauryl sulfate; sodium lauryl sulfoacetate; sodium lauroyl isethionate; sodium laureth carboxylate; sodium dodecyl benzenesulfonate and mixtures thereof. Nonionic surfactants that can be used in the compositions of the present disclosure include, but are not limited to, Polyvinylpyrrolidone (PVP), including various grades of PVP such as PVP K-15, K30, K-60 and K-90, compounds produced by the condensation of alkylene oxide groups with an organic hydrophobic compound which may be aliphatic or alkyl-aromatic in nature. Examples of suitable nonionic surfactants include, but are not limited to, alkyl polyglucosides; block copolymers such as ethylene oxide and propylene oxide copolymers e.g. Poloxamers; ethoxylated hydrogenated castor oils; Alkyl polyethylene oxide e.g. Polysorbates, and/or; fatty alcohol ethoxylates; polyethylene oxide condensates of alkyl phenols; products derived from the condensation of ethylene oxide with the reaction product of propylene oxide and ethylene diamine; ethylene oxide condensates of aliphatic alcohols; long chain tertiary amine oxides; long chain tertiary phosphine oxides; long chain dialkyl sulfoxides; and mixtures thereof. The amphoteric surfactants useful in the compositions of the present disclosure include, but are not limited to, derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Examples of suitable amphoteric surfactants include, but are not limited alkylimino-diproprionates, alkylamphoglycinates (mono or di), alkylamphoproprionates (mono or di), alkylamphoacetates (mono or di), N-alkyl P-aminoproprionic acids, alkylpolyamino carboxylates, phosphorylated imidazolines, alkyl betaines, alkylamido betaines, alkylamidopropyl betaines, alkyl sultaines, alkylamido sultaines, and mixtures thereof. In certain embodiments, the amphoteric surfactant is selected from the group consisting of alkylamidopropyl betaines, amphoacetates such as sodium lauroamphoacetate and mixtures thereof. Mixtures of any of the above mentioned surfactants can also be employed. In an embodiment, the surfactant includes a combination of microcrystalline cellulose and sodium carboxymethyl cellulose such as Vivapur® MCG 811p. Microcrystalline cellulose and/or sodium carboxymethyl cellulose (such as Vivapur® MCG 811p), when used in the composition, may serve a dual function of in situ gel forming polymer and surfactant.

It could be noted during development of the formulation that Fexofenadine HCl has polymorphic nature and has tendency to convert into crystalline form depending upon the solvent used. It could be noted, albeit surprisingly, that this crystal habit can be inhibited or at least slowed down by using a combination of microcrystalline cellulose and sodium carboxymethyl cellulose, which while inhibiting conversion into a crystalline form, also acts as a suspending and stabilizing agent, further retarding the conversion of Fexofenadine into crystalline form.

Accordingly, an aspect of the present disclosure provides a pharmaceutical composition comprising fexofenadine or a salt or a hydrate or a solvate thereof and a zinc salt, said composition being formulated as a liquid formulation meant for nasal administration, wherein said composition comprises a combination of microcrystalline cellulose and sodium carboxymethyl cellulose in an amount ranging from 0.1% w/v to 10% w/v. In an embodiment, the composition includes a combination of microcrystalline cellulose and sodium carboxymethyl cellulose in an amount ranging from 0.1% w/v to 7.5% w/v. Alternatively, the composition includes a combination of microcrystalline cellulose and sodium carboxymethyl cellulose in an amount ranging from 0.1% w/v to 5% w/v. In a preferred embodiment, the composition includes a combination of microcrystalline cellulose and sodium carboxymethyl cellulose in an amount ranging from 0.5% w/v to 5% w/v.

In an embodiment, the composition includes a preservative in an amount ranging from 0.01% w/v to 3% w/v, preferably, ranging from 0.01% w/v to 2% w/v, more preferably, ranging from 0.01% w/v to 1% w/v and most preferably, ranging from 0.01% w/v to 0.5% w/v. Examples of preservatives useful in the compositions of the present disclosure include methyl paraben, propyl paraben, p-hydroxybenzoic acid esters, quaternary ammonium compounds such as benzalkonium chloride, sodium benzoate, benzyl alcohol, butanol, ethanol, isopropyl alcohol and the likes.

During experimentation, it could also be noted that when PEG-400 is used as one of the solvent, it results in higher amounts of RC-A impurities. When propylene glycol (PG) is used in place of PEG-400, it affords dramatic reduction of RC-A impurities as compared to PEG-400. It could also be noted during experimentation that a combination of microcrystalline cellulose and sodium carboxymethyl cellulose (particularly, Vivapur® MCG 811p) tend to lose its viscosity in presence of benzalkonium chloride. Surprisingly, when benzalkonium chloride is replaced with benzyl alcohol, the problem could be resolved.

Accordingly, an aspect of the present disclosure provides a pharmaceutical composition comprising: fexofenadine or a salt or a hydrate or a solvate thereof and a zinc salt, said composition being formulated as a liquid formulation meant for nasal administration, wherein said composition comprises propylene glycol in an amount ranging from 0.1% w/v to 15% w/v. In an embodiment, the composition includes benzyl alcohol in an amount ranging from 0.1% w/v to 10% w/v. In an alternative embodiment, the composition includes benzyl alcohol in an amount ranging from 1% w/v to 10% w/v. In an embodiment, the composition includes benzyl alcohol in an amount ranging from 3% w/v to 13% w/v.

In an embodiment, the amino acids or metabolites or derivatives thereof include(s), but not limited to, glycine, glutamine, asparagine, arginine, lysine in biologically active enantiomeric forms, L-carnitine, choline, betaine, taurine, glycosaminoglycans including hyaluronic acid, chondroitin sulfate, glucosamine, L-glucosamine, heparins and mixtures thereof. In an embodiment, the composition includes hyaluronic acid or salt or derivative thereof in an amount ranging from 0.02% w/v to 3% w/v, preferably, ranging from 0.05% w/v to 2% w/v, more preferably, ranging from 0.1% w/v to 1% w/v and most preferably, ranging from 0.1% w/v to 0.5% w/v.

Examples of stabilizers useful in the compositions of the present disclosure include, but not limited to, gums, agar, and taste masking agents like acrylic polymers, copolymers of acrylates, celluloses, resins and mixtures thereof.

In an embodiment, the fatty acid(s) or derivatives thereof include(s), but not limited to, fatty acids with C1 to C30 carbons, which includes long chain fatty acids; saturated or unsaturated fatty acids and derivatives thereof (monounsaturated fatty acids (MUFAs) C18: ln-12c, C16: ln-5, C16:4n-1 and the polyunsaturated fatty acids (PUFAs) C16:3n-4, C20:3n-3, C20:4n-6, C21:5n-3 and C18:2n-9c, 12t); hydrogenated fatty acids; fatty acid glycerides; polyoxyethylated oleic glycerides; monoglycerides and diglycerides; mono-, bi- or tri-substituted glycerides; glycerol mono-oleate esters; glycerol mono-caprate; glyceryl monocaprylate; dicaprylate; laurate, monolaurate; glyceryl palmitostearate; glyceryl behenate; diethyleneglycol palmitostearate; polyethyleneglycol stearate; polyoxyethyleneglycol palmitostearate; glyceryl mono palmitostearate; cetyl palmitate; polyethyleneglycol palmitostearate; dimethylpolysiloxane; mono- or di-glyceryl behenate; fatty acid derivatives such as diglyceryl lauryl fumarate (DGLF), diglyceryl lauryl succinate, diglyceryl capryl succinate, diglyceryl capryl fumarate; fatty alcohols associated with polyethoxylate fatty alcohols; cetyl alcohol; octyldodecanol; myristyl alcohol; isopropyl myristate, isopropyl palmitate, stearic acid, lauric acid, EPA, DHA, linoleic acid, linolenic acid, stearyl alcohol and mixture thereof. In an embodiment, the fatty acid derivatives includes any or a combination of: diglyceryl lauryl fumarate (DGLF), diglyceryl lauryl succinate, diglyceryl capryl succinate, and diglyceryl capryl fumarate.

The liquid formulation of the present disclosure gets converted into a gel upon its administration in the nasal region and releases the active agent locally in a sustained manner, while providing moisturizing effect and forming a layer on the nasal mucosa that acts like a barrier to entry of pathogens and allergens inhibiting their invasion into sinuses and deep layers of lining of the nose.

While one or more embodiments of the present disclosure enumerates and describes a list of excipients that may be used in the composition to serve an intended purpose, it should be appreciated that one or more excipient may also serve more than one function, obviating the need of inclusion of separate excipient for the specified purpose. For example, a combination of microcrystalline cellulose and sodium carboxymethyl cellulose, when used as part of the composition, while inhibiting conversion of Fexofenadine HCl from amorphous form to crystalline form, it may also serve as one of a surfactant and/or stabilizer, and consequently, it would be apparent to a skilled artisan that one may, in such a case, one can obviate the inclusion of another surfactant and/or stabilizer or can adjust the amount(s) thereof. Although several embodiments of the present disclosure names few of the commonly used excipients, any other excipient known to or appreciated by a skilled person can also be used to realize the advantageous compositions of the present disclosure. Examples of useful excipients which can optionally be added to the composition are described in the Handbook of Pharmaceutical Excipients, 3rd edition, Edited by A. H. Kibbe, Published by: American Pharmaceutical Association, Washington D.C., ISBN: 0-917330-96-X, and in Handbook of Pharmaceutical Excipients (4th edition), Edited by Raymond C Rowe—Publisher: Science and Practice.

The liquid formulation meant for nasal administration of the present disclosure can be prepared by the method that includes: (a) preparing a first premix by taking a part of water (e.g. 40-95% of the total amount of water required in the formulation) in a compounding vessel and mixing a zinc salt and a polyhydric alcohol therewith; (b) preparing a second premix by mixing a surfactant with an aromatic alcohol; (c) adding fexofenadine HCl to the second premix to obtain a fexofenadine solution; (d) adding the Fexofenadine solution to the first premix; and (e) adjusting the final volume with remainder of water.

The compositions realized in accordance with embodiments of the present disclosure can find utility in treatment of a wide variety of allergic conditions/disorders including allergic rhinitis, food allergy, sinusitis, hay fever, asthma and the likes. It could be noted that the components of the compositions realized in accordance with embodiments of the present disclosure exhibit high degree of functional reciprocity, wherein Fexofenadine being a H1 Histamine receptor blocker, affords treatment of allergic conditions/disorders, while zinc salts forms a protective layer on the mucosa.

Accordingly, an embodiment of the present disclosure provides a method of treatment of an allergic condition in a subject, said method comprising administering to a subject in need thereof an effective amount of a composition comprising fexofenadine or a salt or a hydrate or a solvate thereof and a zinc salt. In an embodiment, the composition is formulated as a liquid formulation meant for nasal administration. The allergic condition may be allergic rhinitis, food allergy, sinusitis, hay fever, asthma and the like conditions.

Further embodiment of the present disclosure provides a pharmaceutical composition for use in treatment of an allergic condition, said composition comprising fexofenadine or a salt or a hydrate or a solvate thereof and a zinc salt. The allergic condition may be allergic rhinitis, food allergy, sinusitis, hay fever, asthma and the like conditions.

Yet another embodiment of the present disclosure provides use of a pharmaceutical composition for manufacture of a medicament for treatment of allergic condition, said composition comprising fexofenadine or a salt or a hydrate or a solvate thereof and a zinc salt. The allergic condition may be allergic rhinitis, food allergy, sinusitis, hay fever, asthma and the like conditions.

Further embodiment of the present disclosure provides a pharmaceutical composition for treatment of allergic condition, said composition comprising fexofenadine or a salt or a hydrate or a solvate thereof and a zinc salt. The allergic condition may be allergic rhinitis, food allergy, sinusitis, hay fever, asthma and the like conditions.

The compositions of the present disclosure affords increased therapeutic effects, and reduced adverse effects, making these pharmaceutical compositions extremely effective therapeutics, especially in the treatment of allergic diseases/conditions. Therapeutic levels of the combined drugs will vary from individual to individual and progression stage of disease. The combination medications in the appropriate amounts and intervals effective to treat oral, pharyngeal, oropharyngeal and esophageal disorders or diseases will necessarily be monitored both clinically and chemically by the medical experts or trained physicians.

Further, the patient may receive the specific dosage over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like.

The choice of appropriate dosages for the drugs used in combination therapy according to the present disclosure can be determined and optimized by the skilled artisan, e.g., by observation of the patient, including the patient's overall health, the response to the combination therapy, and the like. Optimization, for example, may be necessary if it is determined that a patient is not exhibiting the desired therapeutic effect or conversely, if the patient is experiencing undesirable or adverse side effects that are too many in number or are of a troublesome severity.

It is especially advantageous to formulate compositions of the present disclosure in unit dosage form for ease of administration and uniformity of dosage. The specifications of the dosage unit forms of the present disclosure are dependent on the unique characteristics of the composition and the particular therapeutic effect to be achieved. Dosages can further be determined by reference to the usual dose and manner of administration of the ingredients. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995).

In certain embodiments, diseases or conditions in the instant invention refers to allergic diseases exposure to allergens induces an IgE mediated inflammation of the mucous membranes lining the nose. The disease manifests symptomatically as nasal congestion, rhinorrhea, itchy nose and sneezing. The nasal mucosa is the primary site for allergen exposure and the inflammatory reactions that cause allergic symptoms. The mechanisms driving the pathophysiology are multifaceted and include activation and migration of effector cells, release of mediators, chemokines and cytokines from inflammatory cells, and damage to the nasal epithelium and nerve endings. Topical administration of the nasal formulation as disclosed in the present invention allow for higher concentrations of drugs to be applied directly to the nasal mucosa the receptor sites of inflammation.

In an embodiment, the nasal formulation disclosed in the present invention acts on histamine receptors to mitigate the effects of histamine by stabilizing the receptor in an inactive state thereby reducing the intrinsic activity of the receptor in response to histamine to reduce symptoms within 30 minutes of nasal administration. The nasal formulation may interfere with the signaling pathway between the H1 receptor and the ubiquitous transcription factor nuclear factor kappa B (NF-κB), which is involved in the expression of pro-inflammatory cytokines, cell adhesion molecules and chemotaxis of inflammatory cells. Further the composition also provides anti-allergic effects by suppressing many stages of the allergic inflammatory reactions which acts on the immune cells such as leukotrienes, prostaglandins, kinins, cytokines, platelet-activating factor (PAF) and ECP, responsible for amplifying inflammation and therefore prolonging symptoms.

In certain embodiments, the disclosed composition may activate the intracellular glucocorticoid receptor that will translocate to the nucleus or interact with transcription factors in the cytoplasm to induced anti-inflammatory effects resulting of the modifications to gene transcription by either increase or suppress the transcription of anti-inflammatory genes and the genes encoding proteins that have inhibitory effects on transcription of inflammatory and immune genes. Thus, the nasal composition may inactivate the activation protein-1 (AP-1) which is responsible for the transcription of many pro-inflammatory genes such as TNF-α, IL-1, IL-2, IL-4, IL-5, IL-6, IL-8, IL-10, IL-13, IFN-γ, GM-CSF

In certain embodiments, the nasal composition may relieve the nasal congestion by activating the postsynaptic α1- and α2 adrenergic receptors on smooth muscles lining nasal vessels. Further the nasal composition exerts antimicrobial action. The composition disrupts the glucose cell-wall transport and intracellular glycolysis thus inhibiting growth of pathogenic bacteria. The nasal composition is an excellent mucolytic reducing the main components of the allergic and inflammatory cascade as well as providing microbiome modulation effects. The micronutrients zinc in the nasal composition modulates antiviral and antibacterial immunity and regulate inflammatory response, the differentiation, proliferation and function of inflammatory cells, by modifying several signaling pathways such as NF-κB signaling pathways and TCR signals. Zinc acts as a mucosal barrier and improve the functions of Th2 cells. Thus, reducing inflammation, and improving mucociliary clearance. Therefore, nasal formulation with fixed dose combination administered topically controls allergic symptoms in the convenient and cost-effective form of a single spray and reduction of symptoms.

Nasal drug delivery devices: Liquid nasal formulations are often aqueous solutions, but suspensions and emulsions can also be delivered. Liquid formulations are considered convenient particularly for topical indications where humidification counteracts the dryness and crusting often accompanies chronic nasal diseases. In traditional spray pump systems, preservatives are typically required to maintain microbiological stability in liquid formulations.

In an embodiment, nasal composition is administered using a nasal delivery device for supplying the composition to a subject's nasal airway, the devices can be a multi-dose or a single-dose. The administration can be done by a mechanical delivery pump, in particular a liquid delivery pump or a powder delivery pump, which delivers metered doses of a substance upon actuation. Aerosol canister may be used for delivering measured volumes of a propellant or similar substance, containing the drug, either as a suspension or as a solution.

In an embodiment, drops may be administered by sucking liquid into a glass dropper, inserting the dropper into a nostril with an extended neck before squeezing the rubber top to emit the drops. Delivery of liquid with rhinyle catheter and squirt tube is a simple way to deposit drug in the nose by inserting tip of a fine catheter or micropipette to the desired area under visual control and squirt the liquid into the desired location. Squeeze bottles can be used by squeezing a partly air-filled plastic bottle, wherein the drug is atomized when delivered from a jet outlet. The dose and particle size vary with the force applied. The metered-dose spray pumps offer high reproducibility of the emitted dose and plume geometry in in-vitro tests. The particle size and plume geometry can vary within certain limits and depend on the properties of the pump, the formulation, the orifice of the actuator, and the force applied. Spray pump may be without preservatives such as a collapsible bag, a movable piston, or a compressed gas to compensate for the emitted liquid volume. The single- and duo-dose spray devices such as metered-dose spray pumps require priming and some degree of overfill to maintain dose conformity for the labeled number of doses. A simple variant of a single-dose spray device (MAD) is a nosepiece with a spray tip fitted to a standard syringe.

In another embodiment, the powered nebulizers and atomizers use compressed gasses (air, oxygen, and nitrogen) or ultrasonic or mechanical power to break up medical solutions and suspensions into small aerosol droplets that can be directly inhaled into the mouth or nose. The smaller particles and slow speed of the nebulized aerosol are advocated to increase penetration to the target sites in the middle and superior meatuses and the paranasal sinuses. Powder medication formulations have greater stability than liquid formulations as preservatives may not be required. Powders tend to stick to the moist surface of the nasal mucosa before being dissolved and cleared. In certain embodiments, bio-adhesive excipients or agents are used for slowing ciliary action to decrease clearance rates and improve absorption. A number of factors like moisture sensitivity, solubility, particle size, particle shape, and flow characteristics will impact deposition and absorption.

Depending on liquid or powder nasal composition, the non-limiting examples of device can include drops delivered with pipette, delivery of liquid with rhinyle catheter and squirt tube, squeeze bottles, metered-dose spray pumps, single- and duo-dose spray devices, nasal pressurized metered-dose inhalers (pMDIs), powered nebulizers and atomizers, VibrENT pulsation membrane nebulizer, aeroneb Solo vibrating mesh nebulizer, ViaNase atomizer, Impel nitrogen-driven atomizer, measured dose aerosol pumps, nasal powder inhalers such as Turbuhaler multi-dose inhaler device modified for nasal inhalation (Rhinocort Turbuhaler® and Blister-based powder inhaler), nasal powder sprayers (such as Fit-lizer™ device, Unidose-DP™, SoluVent™ for intranasal delivery technology), nasal powder insufflators such as rhinyle catheter for liquid delivery by Trimel, Breath-powered Bi-Directional™ technology—nasal drug delivery for liquid and powder medications by OptiNose, single-dose vial irrigation, syringe-irrigation; nasal spray; spray-sol, mucosal atomization device (MAD), rinowash nasal douche and likes. In another embodiment, the device is simple, compact and may readily be carried in the pocket, convenient for use at home or away from home.

### NON-LIMITING EXEMPLARY COMPOSITIONS

Batch ACG001C01310012C and ACG001C01310013C

TABLE 1
Composition for fexofenadine and zinc sulphate nasal spray

| Ingredients | Qty % |
|---|---|
| Fexofenadine. HCL | 0.1 |
| ZnSO4•7H2O | 0.12 |
| Xylitol powder 90 | 2.0 |
| Propylene glycol | 5.0 |
| Benzyl alcohol | 0.9 |
| Polysorbate 20 | 2.5 |
| Glycerol | 5.0 |
| MCC/NaCMC | 2.0 |
| (VIVAPUR MCG 811) | |
| Purified Water | QS to 100% |

Method of preparation of fexofenadine and zinc sulphate nasal spray
i. Premix 1—zinc sulphate solution: 80% water of batch quantity was taken in a compounding vessel and ZnSO4.7H2O, Xylitol and Glycerol were added thereto while mixing for 5 minutes using an overhead stirrer.
ii. Premix 2—propylene glycol, benzyl alcohol and Polysorbate 20 were mixed in another compounding vessel and mixed for 5 minutes using magnetic stirrer at 400 RPM.
iii. Fexofenadine HCl was added to the premix 2 and allowed to dissolve the drug completely.
iv. Solution in compounding vessel 2 (fexofenadine mixed with premix 2) to compounding vessel 1 (premix 1) and allowed to dissolve completely for 30 minutes.
v. The final volume was adjusted with remainder of water and filled into a container.

TABLE 2
Stability data for ACG001C0131012C

| | Assay | RRT | RRT | RRT | |
|---|---|---|---|---|---|
| | Fexo | 1.80 | 3.36 | 3.66 | % |
| Batch No. | HCl | RC A | UK | DCB | Total |
| ACG001C0131012C | 98.8 | 0.02 | 0.03 | 0.05 | 0.119 |

Batch ACG001C0131007B

Nasal spray formulation was prepared using the composition as provided in Table 3 below. Nasal spray formulation was prepared following the same method as described in above example (for batch ACG001C01310012C).

TABLE 3
Composition for fexofenadine and zinc sulphate nasal spray

| Ingredients | Qty % |
|---|---|
| Fexofenadine. HCL | 0.05 |
| ZnSO4•7H2O | 0.12 |
| Xylitol powder 90 | 2.0 |
| Propylene glycol | 5.0 |
| Benzyl alcohol | 0.9 |
| Polysorbate 80 | 0.01 |
| Glycerol | 5.0 |
| MCC/NaCMC | 2.0 |
| (VIVAPUR MCG 811) | |
| Purified Water | QS to 100% |

Batch ACG001C0131006B

Nasal spray formulation was prepared using the composition as provided in Table 4 below. Nasal spray formulation was prepared following the same method as described in above example (for batch ACG001C01310012C).

TABLE 4
Compositions for fexofenadine and zinc sulphate nasal spray

| Ingredients | Qty w/v |
|---|---|
| Fexofenadine. HCL | 0.05% |
| PEG-400 | 20.0% |
| Benzyl alcohol | 0.9% |
| ZnSO4•7H2O | 0.12% |
| Xylitol 90 | 2.0% |
| Tween 80 | 0.01% |
| Glycerol | 5.0% |
| MCC/NaCMC | 2.0% |
| (VIVAPUR MCG 811) | |
| Purified Water | Qs to 100% |

Batch ACG001C0131003B

Nasal spray formulation was prepared using the composition as provided in Table 5 below.

TABLE 5
Composition for fexofenadine and zinc sulphate nasal spray

| Ingredients | Qty w/v |
|---|---|
| Fexofenadine. HCL | 0.05% |
| ZnSO4•7H2O | 0.12% |
| Xylitol Powder | 2.0% |
| PEG-400 | 20.0% |
| Tween 80 | 0.01% |
| Glycerin | 5.0% |
| MCC/NaCMC | 2.0% |
| (VIVAPUR MCG 811) | |
| Water | Qs to 100% |

TABLE 6
Stability data of composition ACG001C0131003B

| | | Assay | RRT | RRT 1.80 | RRT | RRT | % |
|---|---|---|---|---|---|---|---|
| Batch No. | Description | % | 1.58 | RC A | 1.69 | 3.58 | Total |
| ACG001C0131003B | off white | 103.6 | 0.01 | BDL | 0.01 | 0.01 | 0.03 |
| (Nasal liquid) | colour liquid | | | | | | |

Batch ACG001C0131001A

Nasal spray was prepared using the composition as provided in Table 7 below.

TABLE 7
Composition for fexofenadine and zinc sulphate nasal spray

| Ingredients | Qty % |
|---|---|
| Fexofenadine. HCL | 0.05 |
| ZnSO4•7H2O | 0.12 |
| Xylitol powder | 2.0 |
| Propylene glycol | 5.0 |
| Polysorbate 80 | 0.01 |
| Glycerin | 5.0 |
| MCC/NaCMC | 2.0 |
| (VIVAPUR MCG 811) | |
| Purified Water | QS to 100% |

TABLE 8
Stability data for ACG001C0131001A

| | Description | | | | | | |
|---|---|---|---|---|---|---|---|
| | Off white colour viscous liquid with free from undissolved material | | | | | | |
| | | | 1 Month | 2 Month | 3 Month | 3 Month | 3 Month |
| | Proposed | | 40° C./ | 40° C./ | 40° C./ | 30° C./ | 25° C./ |
| Test parameters | Specification | Initial | 75% RH | 75% RH | 75% RH | 65% RH | 60% RH |
| Assay- | 90.0-110.0% | 101.6 | 102.5 | 101.6 | 102.5 | 102.0 | 103.0 |
| Fexofenadine | | | | | | | |
| HCl | | | | | | | |

Related substances. (%, w/v)

| | | | | | | | |
|---|---|---|---|---|---|---|---|
| Related | NMT 0.4% | ND | ND | 0.011 | 0.023 | 0.019 | 0.017 |
| Compound A | | | | | | | |
| Decarboxylated | NMT 0.2% | 0.041 | 0.031 | 0.041 | 0.025 | 0.030 | 0.027 |
| degradant | | | | | | | |
| Any Unspecified | NMT 0.2% | 0.012 | 0.021 | 0.015 | 0.025 | 0.021 | 0.011 |
| degradant | | | | | | | |
| Total | NMT 1.5% | 0.052 | 0.052 | 0.067 | 0.080 | 0.070 | 0.055 |
| degradants | | | | | | | |

Nasal spray formulations were prepared using the composition as provided in Table 9 below.

TABLE 9
Compositions for fexofenadine and zinc sulphate nasal spray

| Ingredients | Qty w/v | Qty w/v | Qty w/v | Qty w/v |
|---|---|---|---|---|
| Fexofenadine. HCL | 0.05% | 0.05% | 0.05% | 0.05% |
| ZnSO4•7H2O | 0.12% | 0.12% | 0.12% | 0.12% |
| Xylitol Powder | 2.0% | 2.0% | 2.0% | — |
| PEG-400 | 20.0% | — | 20.0% | 20.0% |
| Propylene Glycol | 5.0% | 5.0% | — | — |
| Benzyl Alcohol | 0.5% | 0.5% | 0.5% | — |
| Tween 80 | 0.01% | 0.01% | 0.01% | 0.01% |
| Glycerin | 5.0% | 5.0% | 5.0% | 5.0% |
| MCC/NaCMC | 2.0% | 2.0% | 2.0% | 2.0% |
| (VIVAPUR MCG 811) | | | | |
| Water | Qs to 100% | Qs to 100% | Qs to 100% | Qs to 100% |

Batch ACG001C0131004B

Nasal spray formulation was prepared using the composition as provided in Table 10 below.

TABLE 10
Composition for fexofenadine and zinc sulphate nasal spray

| Ingredients | Qty w/v |
|---|---|
| Fexofenadine. HCL | 0.05% |
| ZnSO4•7H2O | 0.12% |
| Xylitol Powder | 2.0% |
| PEG-400 | 20.0% |
| Tween 80 | 0.01% |
| Glycerin | 5.0% |
| MCC/NaCMC | 2.0% |
| (VIVAPUR MCG 811) | |
| Water | Qs to 100% |

Batch ACG001C0131005B

Nasal spray formulation was prepared using the composition as provided in Table 11 below.

TABLE 11
Composition for fexofenadine and zinc sulphate nasal spray

| Ingredients | Qty w/v |
|---|---|
| Fexofenadine. HCL | 0.05% |
| ZnSO4•7H2O | 0.12% |
| Xylitol 90 | 2.0% |
| PEG-400 | 20.0% |
| Benzyl alcohol | 0.9% |
| Benzalkonium Chloride | 0.04% |
| Tween 80 | 0.01% |
| Glycerol | 5.0% |
| MCC/NaCMC | 2.0% |
| (VIVAPUR MCG 811) | |
| Water | Qs to 100% |

Nasal spray formulation was prepared using the composition as provided in Table 12 below.

TABLE 12
Composition for fexofenadine nasal spray

| Ingredients | Qty w/v |
|---|---|
| Fexofenadine. HCL | 0.05% |
| PEG-400 | 20.0% |
| Benzyl alcohol | 0.9% |
| ZnSO4•7H2O | 0.12% |
| Xylitol 90 | 2.0% |
| Tween 80 | 0.01% |
| Glycerol | 5.0% |
| Dibasic sodium phosphate | 0.02065% |
| Monobasic sodium phosphate | 0.343% |
| MCC/NaCMC | 2.0% |
| (VIVAPUR MCG 811) | |
| Purified Water | Qs to 100% |

Batch ACG001C0131009B

Nasal spray formulation was prepared using the composition as provided in Table 13 below.

TABLE 13
Composition for fexofenadine nasal spray

| Ingredients | Qty w/v |
|---|---|
| Fexofenadine Hydrochloride | 0.05% |
| PG | 5.0% |
| ZnSO4•7H2O | 0.12% |
| Xylitol 90 | 2.0% |
| Tween 80 | 0.01% |
| Glycerol | 5.0% |
| MCC/NaCMC | 2.0% |
| (VIVAPUR MCG 811) | |
| Purified Water | Qs to 100% |

Batch ACG001C01310011B

Nasal spray formulation was prepared using the composition as provided in Table 14 below.

TABLE 14
Composition for fexofenadine and zinc sulphate nasal spray

| Ingredients | Qty % |
|---|---|
| Fexofenadine. HCL | 0.2 |
| ZnSO4•7H2O | 0.12 |
| Xylitol powder 90 | 2.0 |
| Propylene glycol | 5.0 |
| Benzyl alcohol | 0.9 |
| Polysorbate 20 | 2.5 |
| Glycerol | 5.0 |
| MCC/NaCMC | 2.0 |
| (VIVAPUR MCG 811) | |
| Purified Water | QS to 100% |

Batch ACG001C01310015C

Nasal spray formulation was prepared using the composition as provided in Table 15 below.

TABLE 15
Composition for fexofenadine and zinc sulphate nasal spray

| Ingredients | Qty % |
|---|---|
| Fexofenadine. HCL | 0.4 |
| ZnSO4•7H2O | 0.12 |
| Xylitol powder 90 | 2.0 |
| PEG-400 | 20.0 |
| Benzyl alcohol | 0.5 |
| Polysorbate 20 | 2.5 |
| Glycerol | 5.0 |
| MCC/NaCMC | 2.0 |
| (VIVAPUR MCG 811) | |
| Purified Water | QS to 100% |

Batch ACG001C01310016C

Nasal spray formulation was prepared using the composition as provided in Table 16 below.

TABLE 16
Composition for fexofenadine and zinc sulphate nasal spray

| Ingredients | Qty % |
|---|---|
| Fexofenadine. HCL | 0.4 |
| ZnSO4•7H2O | 0.12 |
| Xylitol powder 90 | 2.0 |
| PEG-400 | 20.0 |
| Benzyl alcohol | 0.9 |
| Polysorbate 20 | 2.5 |
| Glycerol | 2.5 |
| MCC/NaCMC | 2.0 |
| (VIVAPUR MCG 811) | |
| Purified Water | QS to 100% |

TABLE 17
DRUG-EXCIPIENT COMPATABILITY STUDY RESULTS
ECS Results 40° C./75% RH-4 W

| | | | | | % Degradants | | | | |
|---|---|---|---|---|---|---|---|---|---|
| | | | | | Specified | | | | |
| | | Drug | | | RRT | RRT | Unspecified | | |
| | Drug + | Excipient | Time | Analysis | 1.80 | 3.36 | Individual | % | |
| Batch No. | Excipient | Ratio | period | date | RCA | DCB | Highest | Total | Description |
| In House Specifications | | | | | 0.4 | 0.2 | 0.2 | 2.5 | — |
| ACG001C0131014 | API | — | Initial | 18 Jul. 2021 | 0.02 | 0.02 | 0.04 | 0.09 | Clear |
| | | | | | | | | | solution |
| | | | 40° C./75% | 13 Aug. 2021 | 0.01 | 0.02 | 0.04 | 0.11 | Clear |
| | | | RH-4 W | | | | | | solution |
| | Drug + | 1:125 | Initial | 18 Jul. 2021 | 0.05 | 0.04 | 0.06 | 0.13 | Clear |
| | PEG400 + | | | | | | | | solution |
| | Water | | 40° C./75% | 13 Aug. 2021 | 0.32 | 0.04 | 0.06 | 0.46 | Clear |
| | | | RH-4 W | | | | | | solution |
| | Drug + | 1:31.25 | Initial | 18 Jul. 2021 | 0.03 | 0.03 | 0.07 | 0.12 | Clear |
| | Water + PG | | | | | | | | solution |
| | | | 40° C./75% | 13 Aug. 2021 | 0.06 | 0.03 | 0.05 | 0.23 | Clear |
| | | | RH-4 W | | | | | | solution |
| | Drug + | 1:5.625 | Initial | 18 Jul. 2021 | 0.08 | 0.04 | 0.05 | 0.11 | Clear |
| | Water + | | | | | | | | solution |
| | Benzyl | | 40° C./75% | 13 Aug. 2021 | 0.20 | 0.03 | 0.08 | 0.38 | Clear |
| | alcohol | | RH-4 W | | | | | | solution |
| | Drug + | 1:31.25 | Initial | 18 Jul. 2021 | 0.02 | 0.03 | 0.05 | 0.11 | Clear |
| | Water + | | | | | | | | solution |
| | Glycerol | | 40° C./75% | 13 Aug. 2021 | 0.03 | 0.03 | 0.05 | 0.15 | Clear |
| | anhydrous | | RH-4 W | | | | | | solution |
| | Drug + | 1:15.625 | Initial | 18 Jul. 2021 | 0.12 | 0.03 | 0.05 | 0.20 | Clear |
| | Water + | | | | | | | | solution |
| | Polysorbate 20 | | 40° C./75% | 13 Aug. 2021 | 4.20 | 0.03 | 0.06 | 4.41 | Clear |
| | | | RH-4 W | | | | | | solution |
| | Drug + | 1:0.0625 | Initial | 18 Jul. 2021 | 0.05 | 0.03 | 0.05 | 0.13 | Clear |
| | Water + | | | | | | | | solution |
| | Polysorbate 80 | | 40° C./75% | 13 Aug. 2021 | 2.80 | 0.03 | 0.31 | 3.74 | Clear |
| | | | RH-4 W | | | | | | solution |
| | Drug + | 1:12.5 | Initial | 18 Jul. 2021 | 0.03 | 0.02 | 0.05 | 0.09 | Clear |
| | Water + | | | | | | | | solution |
| | MCC/NaCMC | | 40° C./75% | 13 Aug. 2021 | 0.06 | 0.03 | 0.05 | 0.21 | Clear |
| | | | RH-4 W | | | | | | solution |
| | Drug + | 1:12.5 | Initial | 18 Jul. 2021 | 0.02 | 0.03 | 0.07 | 0.14 | Clear |
| | Water + | | | | | | | | solution |
| | Xylitol 90 | | 40° C./75% | 13 Aug. 2021 | 0.02 | 0.04 | 0.06 | 0.21 | Clear |
| | | | RH-4 W | | | | | | solution |
| | Drug + | 1:0.75 | Initial | 18 Jul. 2021 | 0.02 | 0.03 | 0.05 | 0.12 | Clear |
| | Water + | | | | | | | | solution |
| | Zinc sulphate | | 40° C./75% | 13 Aug. 2021 | 0.04 | 0.04 | 1.39 | 1.74 | Clear |
| | | | RH-4 W | | | | | | solution |
| | Drug + | — | Initial | 18 Jul. 2021 | 0.02 | 0.03 | 0.07 | 0.15 | Clear |
| | water | | | | | | | | solution |
| | | | 40° C./75% | 13 Aug. 2021 | 0.02 | 0.04 | 0.07 | 0.15 | Clear |
| | | | RH-4 W | | | | | | solution |
| | Mixture-1 | — | Initial | 18 Jul. 2021 | 0.03 | 0.03 | 0.05 | 0.10 | Clear |
| | drug + PG + | | | | | | | | solution |
| | Glycerol + | | 40° C./75% | 13 Aug. 2021 | 0.02 | 0.04 | 0.07 | 0.13 | Clear |
| | PS 80 + | | RH-4 W | | | | | | solution |
| | MCC/NaCMC + | | | | | | | | |
| | Xylitol 90 + | | | | | | | | |
| | Zinc sulphate + | | | | | | | | |
| | water | | | | | | | | |
| | Mixture-2 | — | Initial | 18 Jul. 2021 | 0.03 | 0.03 | 0.05 | 0.10 | Clear |
| | drug + PG + | | | | | | | | solution |
| | Glycerol + | | 40° C./75% | 13 Aug. 2021 | 0.09 | 0.03 | 0.07 | 0.20 | Clear |
| | PS 80 + | | RH-4 W | | | | | | solution |
| | MCC/NaCMC + | | | | | | | | |
| | Xylitol 90 + | | | | | | | | |
| | Zinc sulphate + | | | | | | | | |
| | water | | | | | | | | |
| | Mixture-3 | — | Initial | 18 Jul. 2021 | 0.03 | 0.03 | 0.06 | 0.17 | Clear |
| | drug + PG + | | | | | | | | solution |
| | Glycerol + | | 40° C./75% | 13 Aug. 2021 | 0.05 | 0.03 | 0.06 | 0.15 | Clear |
| | PS 80 + | | RH-4 W | | | | | | solution |
| | MCC/NaCMC + | | | | | | | | |
| | Xylitol 90 + | | | | | | | | |
| | Zinc sulphate + | | | | | | | | |
| | water | | | | | | | | |
| | Mixture-4 | — | Initial | 18 Jul. 2021 | 0.03 | 0.03 | 0.05 | 0.10 | Clear |
| | drug + PG + | | | | | | | | solution |
| | Glycerol + | | 40° C./75% | 13 Aug. 2021 | 0.02 | 0.03 | 0.06 | 0.11 | Clear |
| | PS 80 + | | RH-4 W | | | | | | solution |
| | MCC/NaCMC + | | | | | | | | |
| | Xylitol 90 + | | | | | | | | |
| | Zinc sulphate + | | | | | | | | |
| | water | | | | | | | | |
| | Mixture-5 | — | Initial | 18 Jul. 2021 | 0.03 | 0.04 | 0.06 | 0.13 | Clear |
| | drug + PG + | | | | | | | | solution |
| | Glycerol + | | 40° C./75% | 13 Aug. 2021 | 0.03 | 0.03 | 0.06 | 0.134 | Clear |
| | PS 80 + | | RH-4 W | | | | | | solution |
| | MCC/NaCMC + | | | | | | | | |
| | Xylitol 90 + | | | | | | | | |
| | Zinc sulphate + | | | | | | | | |
| | water | | | | | | | | |

Nasal spray formulation was prepared using the composition as provided below.

TABLE 18
Composition for fexofenadine, corticosteroid
and zinc sulphate nasal spray

| Ingredients | Qty % |
|---|---|
| Fexofenadine. HCL | 0.2 |
| Fluticasone Furoate | 0.0012 |
| ZnSO4•7H2O | 0.12 |
| Xylitol powder 90 | 2.0 |
| Propylene glycol | 5.0 |
| Benzyl alcohol | 0.9 |
| Polysorbate 20 | 2.5 |
| Glycerol | 5.0 |
| MCC/NaCMC | 2.0 |
| (VIVAPUR MCG 811) | |
| Purified Water | QS to 100% |

Characterization Data:

### Batch No: ACG001C0131007B

pH: The pH of the formulation is measured using pH meter (Mettler Toledo, seven compact S210) and value was found to be 4.94±0.11
Density: Density was estimated using densitometer (Mettler Toledo, Density 2Go). The density was found to be 1.02 g/cm³

### Batch No: ACG001C01310011B

pH: The pH of the formulation is measured using pH meter (Mettler Toledo, Seven compact S210). The pH was found to be 4.51±0.04
Density: Density was estimated using densitometer (Mettler Toledo, Density 2Go). The density was found to be 1.02 g/cm³

### Batch No: ACG001C01310013C

pH: The pH of the formulation is measured using pH meter (Mettler Toledo, Seven compact S210) and value was found to be 4.79±0.09
Density: Density was estimated using densitometer (Mettler Toledo, Density 2Go). The density was found to be 1.02 g/cm³

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

What is claimed is:

1. A pharmaceutical composition comprising fexofenadine, or a salt or a hydrate or a solvate thereof, and a zinc salt, wherein said composition is formulated as a liquid formulation for nasal administration.

2. The pharmaceutical composition of claim 1, wherein the pharmaceutical composition comprises fexofenadine, or a salt or a hydrate or a solvate thereof, and a zinc salt in a weight ratio ranging from 1:1 to 1:20.

3. The pharmaceutical composition of claim 1, wherein the pharmaceutical composition comprises fexofenadine, or a salt or a hydrate or a solvate thereof, and a zinc salt in a weight ratio ranging from 1:1.5 to 1:10.

4. The pharmaceutical composition of claim 1, wherein the pharmaceutical composition further comprises at least one excipient.

5. The pharmaceutical composition of claim 1, wherein the pharmaceutical composition further comprises at least one other active agent selected from the group consisting of a corticosteroid, a 5-HT1 agonists, an ergolines, a TCA, an anticonvulsant, an antihistamine, an anti-allergy agent, an aminothiol, an anti-inflammatory agent, immunosuppressant, a nitric oxide releasing drug, a PDE inhibitor, a JAK inhibitor, a TCA, a bronchodilator and any combination thereof.

6. The pharmaceutical composition of claim 4, wherein the at least one excipient is selected from the group consisting off a diluent, an antioxidant, a preservative, a solvent, a polyhydric alcohol, a sugar alcohol, a fatty acid or derivative thereof, an amino acid or metabolite or derivative thereof, a surfactant, a solubilizer, a stabilizer and any combination thereof.

7. The pharmaceutical composition of claim 1, wherein the pharmaceutical composition comprises
fexofenadine or a salt or a hydrate or a solvate thereof in an amount ranging from 0.02% w/v to 2% w/v;
a zinc salt in an amount ranging from 0.02% w/v to 5% w/v;
a polyhydric alcohol in an amount ranging from 5% w/v to 30% w/v;
a surfactant in an amount ranging from 1% w/v to 30% w/v;
a sugar alcohol in an amount ranging from 0.5% w/v to 25% w/v; and
water in an amount ranging from 35% w/v to 90% w/v.

8. The pharmaceutical composition of claim 7, wherein the polyhydric alcohol is selected from the group consisting of polyhydric alkanes, polyhydric alkane esters, polyalkene glycols, and any mixtures thereof.

9. The pharmaceutical composition of claim 7, wherein the sugar alcohol is selected from the group consisting of sorbitol, xylitol, mannitol, maltitol, inositol, allitol, altriol, dulcitol, galactitol, glucitol, hexitol, iditol, pentitol, ribitol, erythritol, and any mixtures thereof.

10. The pharmaceutical composition of claim 7, wherein the pharmaceutical composition comprises a preservative in an amount ranging from 0.01% w/v to 5% w/v, and wherein said preservative is benzyl alcohol.

11. The pharmaceutical composition of claim 7, wherein the surfactant comprises a combination of microcrystalline cellulose and sodium carboxymethyl cellulose.

12. A pharmaceutical composition of claim 1, wherein said liquid formulation turns into a gel upon nasal administration.

13. A pharmaceutical composition of claim 1, wherein said composition comprises a combination of microcrystalline cellulose and sodium carboxymethyl cellulose in an amount ranging from 0.1% w/v to 10% w/v.

14. A pharmaceutical composition of claim 1, wherein said composition comprises propylene glycol in an amount ranging from 0.1% w/v to 15% w/v.

15. The pharmaceutical composition of claim 5, wherein the other active agents is selected from a group consisting of beclomethasone, budesonide, ciclesonide, flunisolide, fluticasone furoate, fluticasone propionate, mometasone, triamcinolone, prednisone, desloratadine, azelastine, cetirizine, terfenadine, chlorphenamine, levocetirizine, montelukast, loratadine, bilastine, levalbuterol, olopatadine, brompheniramine, benralizumab, chlorpheniramine, clemastine, cromolyn, cyproheptadine, ibuprofen, diphenhydramine, hydroxyzine, promethazine, triprolidine, ketotifen, naphazoline, pheniramine, methylprednisolone, dexamethasone, pseudoephedrine, phenylephrine, albuterol, ipratropium bromide, vilanterol, salbutamol, salmeterol, formoterol, oxymetazoline, xylometazoline, amidrine, fluticasone, glycopyrronium, tiotropium, arformoterol, theophylline, aminophylline, ipratropium, bitolterol, carbuterol, fenoterol, isoetarine, pirbuterol, procaterol, reproterol, rimiterol, salbutamol, levosalbutamol, terbutaline, tulobuterol, bambuterol, clenbuterol, formoterol/arformoterol, salmeterol, salmefamol, abediterol, carmoterol, indacaterol, olodaterol, vilanterol, epinephrine, hexoprenaline, isoprenaline (isoproterenol), orciprenaline (metaproterenol), beclomethasone, budesonide, ciclesonide, aclidinium bromide, ipratropium bromide, oxitropium bromide, tiotropium bromide, umeclidinium bromide, acefylline, ambuphylline, aminophylline, bamifylline, choline theophyllinate, caffeine, doxofylline, enprofylline, etamiphylline, proxyphylline, theophylline, pranlukast, zafirlukast, zileuton, ramatroban, seratrodast, cysteamine HCl, azathioprine, mycophenolic acid, leflunomide, teriflunomide, ciclosporin, pimecrolimus, tacrolimus, voclosporin, lenalidomide, pomalidomide, thalidomide, apremilast, sirolimus, everolimus, ridaforolimus, temsirolimus, umirolimus, zotarolimus, baricitinib, blisibimod, nilotinib, filgotinib, tofacitinib, upadacitinib, abatacept, belatacept, etanercept pegsunercept, aflibercept, alefacept, rilonacept, glyceryl trinitrate, isosorbide dinitrate, isosorbide mononitrate, isoamyl nitrite, almotriptan, avitriptan, eletriptan, frovatriptan, naratriptan, rizatriptan, sumatriptan, zolmitriptan, dihydroergocryptine, dihydroergotamine, ergotamine, lisuride methylergometrine, methysergide, amitriptyline, nortriptyline, imipramine, carbamazepine, oxcarbazepine, topiramate, valproate, apremilast, arofylline, atizoram, benafentrine, catramilast, CC-1088, CDP-840, CGH-2466, cilomilast, cipamfylline, crisaborole, denbutylline, difamilast, drotaverine, etazolate, filaminast, glaucine, HT-0712, ICI-63197 indimilast, irsogladine, lavamilast, lirimilast, lotamilast, luteolin, mesembrenone, mesembrine, mesopram, oglemilast, piclamilast, pumafentrine, revamilast, Ro 20-1724, roflumilast, rolipram, ronomilast, RPL-554, RS-25344, tetomilast, tofimilast, YM-976, zardaverine, ibudilast, roflumilast, adibendan, amrinone (inamrinone), anagrelide, benafentrine, bucladesine, carbazeran, cilostamide, cilostazol, enoximone, imazodan, KMUP-1, meribendan, milrinone, olprinone, parogrelil, pimobendan, pumafentrine, quazinone, RPL-554, siguazodan, trequinsin, vesnarinone, zardaverine, acetildenafil, aildenafil, avanafil, beminafil, benzamidenafil, dasantafil, icariin, gisadenafil, homosildenafil, lodenafil, mirodenafil, MY-5445, nitrosoprodenafil, norcarbodenafil, SCH-51866, sildenafil, sulfoaildenafil, T-0156, tadalafil, udenafil, vardenafil, abrocitinib, baricitinib, filgotinib, momelotinib, oclacitinib, peficitinib, ruxolitinib, tofacitinib, tasocitinib, CP-690550, upadacitinib, atiprimod, AZD-1480, baricitinib, chz868, cucurbitacin I (elatericin B, JSI-124) CYT387 lestaurtinib, NSC-7908, NSC-33994, pacritinib, peficitinib, ruxolitinib, SD-1008, cercosporamide, decernotinib (VX-509), peficitinib, TCS-21311, WHI-P 15 ZM-39923, ZM-449829, other derivatives thereof, analogs thereof, and any combinations thereof.

16. A method of treating an allergic condition in a patient in need thereof comprising administering to a subject a therapeutically effective amount of the pharmaceutical composition of claim 1.

17. Use of the pharmaceutical composition of claim 1 in preparation of a medicament for the treatment of an allergic condition in patient in need thereof.

18. A method for manufacturing the pharmaceutical composition of claim 1 comprising the steps of:
a. preparing a premix 1, wherein the premix 1, comprises of adding ZnSO4.7H2O, xylitol, glycerol, and water, in a compounding vessel to obtain a mixture using an overhead stirrer or equivalent;
b. preparing a premix 2, wherein the premix 2, comprises of propylene glycol, benzyl alcohol and polysorbate 20 or polysorbate 80 is selected and is mixed in another compounding vessel using magnetic stirrer or equivalent;
c. fexofenadine HCl is added to the premix 2 and allowed to dissolve completely;
d. solution from step (c) is mixed into premix 1 from step (a) and allowed to dissolve completely; and
e. final volume of the nasal composition from step (d) is adjusted with water.
